(19) United States
(12) Patent Application Publication
(10) Pub. No.: US 2017/0336380 A1
(43) Pub. Date: Nov. 23, 2017

(54) PORTABLE FLUSHING MONITOR

(71) Applicant: John C. Kupferle Foundry Company, St. Louis, MO (US)

(72) Inventors: Daniel G. McKeague, Kirkwood, MO (US); Daniel C. Lorentz, St. Louis, MO (US)

(21) Appl. No.: 15/602,325

(22) Filed: May 23, 2017

Related U.S. Application Data
(60) Provisional application No. 62/505,307, filed on May 12, 2017.
(60) Provisional application No. 62/340,178, filed on May 23, 2016.

(51) Int. Cl. G01N 33/18; E03B 7/07; E03B 7/00; E03B 9/14; G08B 21/18; G01N 1/20

(52) U.S. Cl. CPC G01N 33/18; G08B 21/182; G01N 1/2035; E03B 7/006; E03B 9/14; E03B 7/07; G01N 2001/205

(57) ABSTRACT

A device for monitoring manual flushing of a hydrant includes an adapter attached to a flushing hose and attachable to an outlet of a water hydrant, an access port on the adapter, the access port being sized to take a sample of a stream of water flowing through the adapter, a flexible hose attached to the access port, an analyzer box containing a sensor adapted to sense a characteristic of water, the sensor being operatively connected to the flexible hose, and an alarm operatively attached to the sensor.

### CROSS-REFERENCE TO RELATED APPLICATIONS

This application claims priority to U.S. provisional applications Ser. No. 62/340,178, filed May 23, 2016, and Ser. No. 62/505,307, filed May 12, 2017. The disclosures of both these applications are hereby incorporated by reference.

### STATEMENT REGARDING FEDERALLY SPONSORED RESEARCH

Not applicable.

### BACKGROUND OF THE INVENTION

Monitoring of subterranean water systems, such as municipal drinking water systems, is a longstanding and increasingly important task. For example, dead-ends on water systems are a constant source of water quality problems. The water is not flowing and therefore becomes stagnant, and flushing is required. Even in mains which are in use, monitoring of water quality is required, and flushing of hydrants, either dedicated flushing hydrants or fire hydrants, is required on a regular basis.

A solution frequently used when flushing is required on a schedule measured in hours or days is to mount an automatic flushing hydrant at the dead-end or elsewhere in the main. The automatic flusher discharges water for an interval determined either by a timer or a water condition sensor such as a chlorine sensor. An example of such a system is shown in McKeague, U.S. Pat. No. 8,733,390. In temperate climates that include freezing temperatures, the automatic flushing hydrant must be buried below the frost line. This makes such hydrants expensive and difficult to access.

Most hydrants in such systems are above ground and include a subterranean shutoff valve, leaving the above-ground portions of the hydrant dry. These hydrants are sometimes flushed by attaching a portable automatic flusher to the hydrant, as shown, for example, in McKeague, U.S. Pat. No. 6,820,635. These attachments are somewhat expensive. They also require opening the hydrant's subterranean valve, thus leaving them “wet” and subject to freezing. If a hydrant with a portable automatic flusher is needed for fire-fighting, the hydrant valve must first be closed to permit the fire hose to be attached, and may require removal of the portable flusher if it is in the direction of the fire. Further, the portable flushing devices are left unattended, and they are therefore subject to accidental damage and vandalism. They must also be moved periodically from hydrant to hydrant.

For hydrants which require flushing less often, or in winter months in temperate climates, or when capital budgets do not permit purchase of numerous automatic flushing systems for hydrants, most hydrants are flushed manually. Manual flushing may also be carried out periodically in order to provide visual inspection of hydrants and their operation. A person or crew (“operator” herein) is sent to each hydrant. The operator removes a cap from the hydrant outlet fitting and usually attaches a drain hose to the hydrant outlet fitting. The operator then uses a wrench to manually turn an accessible stem of the hydrant's subterranean valve, and the hydrant is flushed until the water's color and odor seem acceptable. A sample is then frequently taken and tested with a hand-held testing device to confirm that the level of chlorine or some other water characteristic is at an acceptable level. A sample is sometimes taken back to a lab to be tested, to assure that flushing was complete and the water quality is acceptable at that location. Whether or not a sample is taken, flushing is continued for an extended period of time to ensure a high probability that the water emerging from the hydrant is of acceptable quality. The hydrant valve is then closed, the drain hose removed from the fitting, and the cap replaced. The process is time-consuming and uses a considerable quantity of water at each hydrant. The information obtained at each hydrant is subject to whatever subjective report the operator may convey and is generally sketchy.

### BRIEF SUMMARY OF THE INVENTION

In accordance with the present invention a portable device is provided for real-time monitoring of water being manually flushed from a hydrant. In embodiments, an analyzer in the device activates an alarm when a predetermined water quality level is reached. The “alarm” may be any device or system that alerts the operator the flushing cycle is complete and the hydrant should be closed. In some embodiments, chlorine levels are measured. In other embodiments, temperature, pH, pressure, turbidity, and other characteristics are measured. It will be understood that the device may measure and/or log multiple parameters and may trigger the alarm in response to some combination of parameters. In some embodiments, a data logger is provided and may produce a record of such parameters as flush duration, chlorine (or other parameter) residual level against time, and initial versus final free chlorine (or other parameter). In some embodiments a clock records start and finish times. In some embodiments the data logger function is incorporated in a programmable logic controller (PLC). A sampling valve may be provided for manually or automatically drawing water samples for immediate or laboratory testing of such parameters as bacteria and organic chlorine.

### BRIEF DESCRIPTION OF THE SEVERAL VIEWS OF THE DRAWINGS

FIG. 1 is a somewhat diagrammatic view in side elevation showing an embodiment of the portable flushing monitor of the present invention attached to a hydrant.

FIG. 2 is a top plan view of an analyzer box portion of the flushing monitor of FIG. 1, with a lid of the box removed.

FIG. 3 is a bottom plan view of a shelf portion of the analyzer box of

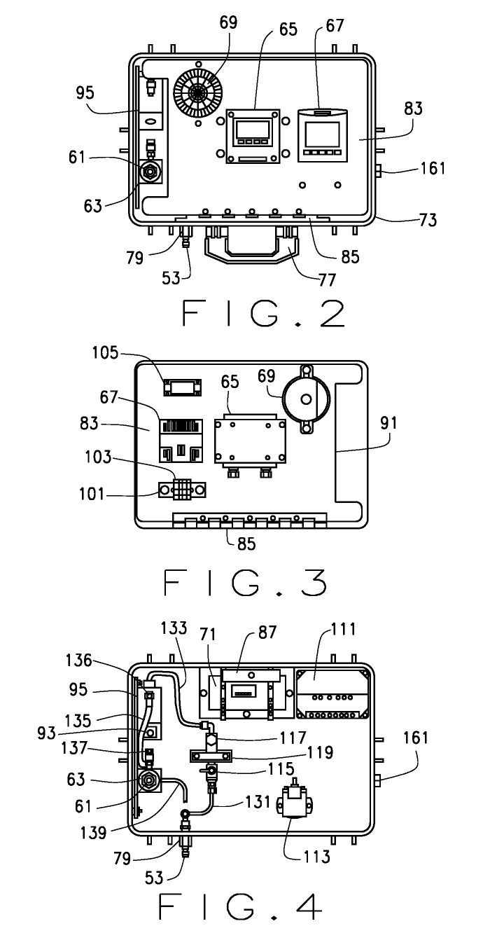

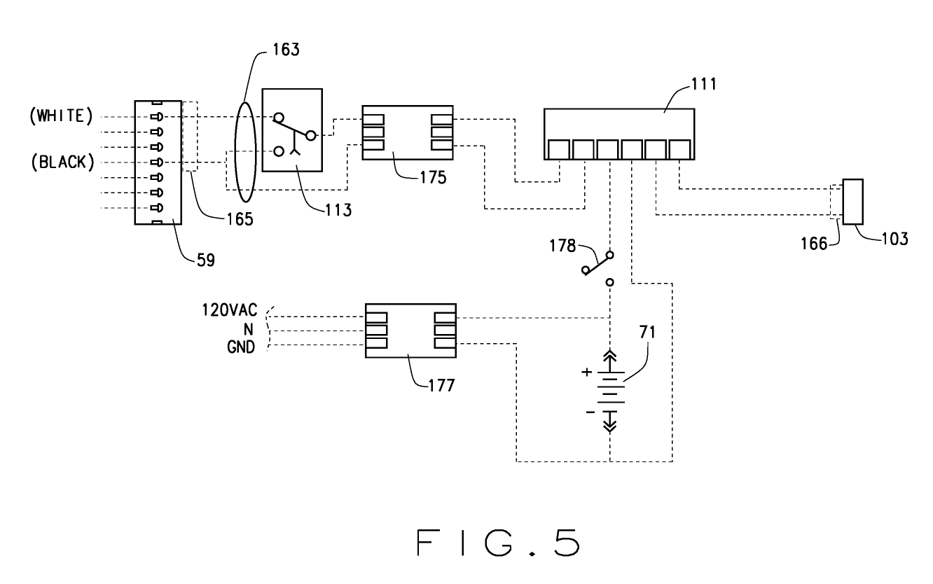

FIG. 2.

FIG. 4 is a top plan view of the analyzer box of FIG. 2, with a shelf portion removed.

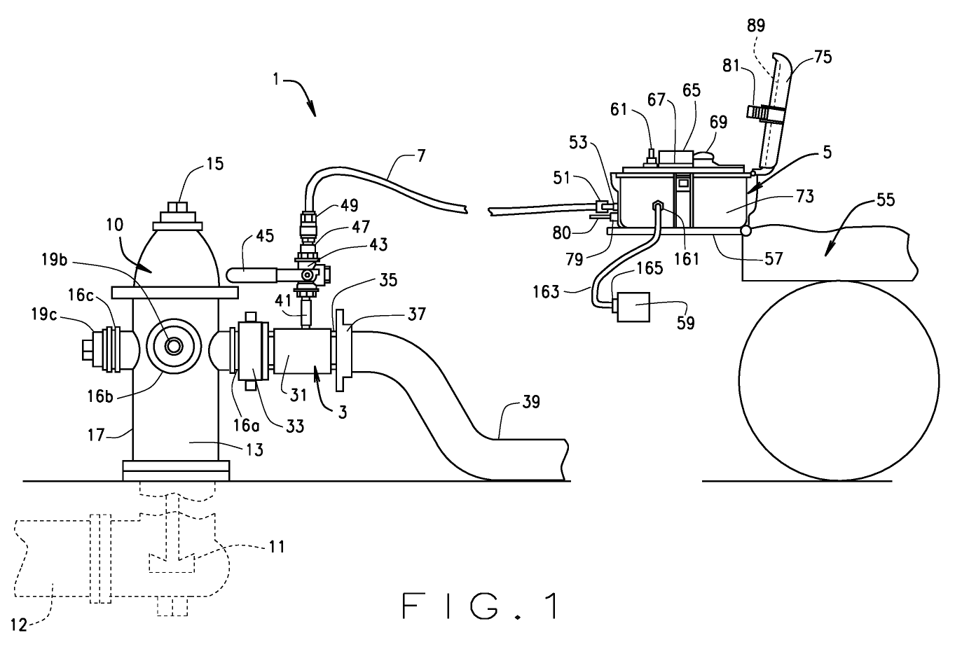

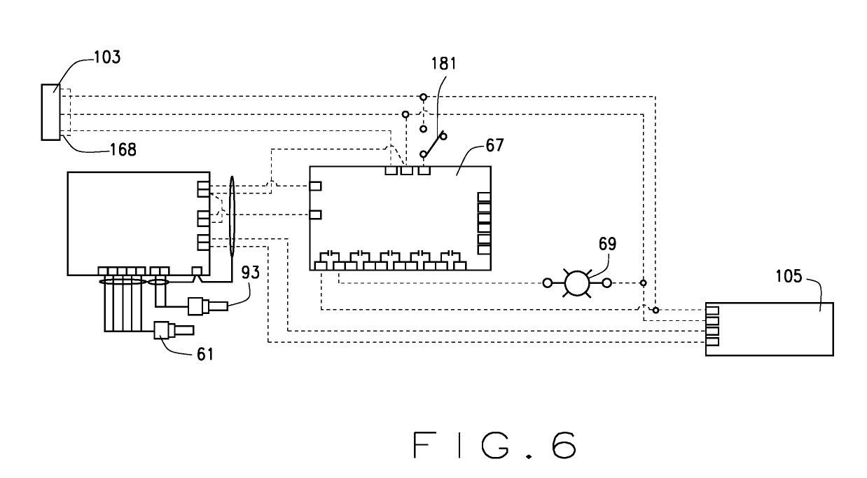

FIG. 5 is a wiring diagram of an embodiment of a power supply for the flushing monitor of FIGS. 1-4.

FIG. 6 is a wiring diagram of the flushing monitor of FIGS. 1-5.

### DESCRIPTION OF THE PREFERRED EMBODIMENT(S)

The following detailed description illustrates the invention by way of example and not by way of limitation. The description clearly enables one skilled in the art to make and use the invention, describes several embodiments, adaptations, variations, alternatives, and uses of the invention, including what is presently believed to be the best mode of carrying out the invention.

As shown in FIG. 1, an illustrative embodiment of a portable flushing monitor 1 for monitoring manual flushing of a hydrant includes an adapter 3, an analyzer enclosure 5, and a flexible sampling hose 7 connecting the adapter 3 and the box 5. The adapter 3 is attached to one outlet of a hydrant 10.

The hydrant 10 is illustratively a so-called dry barrel hydrant, having a valve 11 below ground, generally below the local frost line, connecting the hydrant to a municipal water distribution system indicated generally at 12. The valve 11 is self-draining, so that, when it is closed, water drains from the cast body 13 of the hydrant 10. The valve 11 is opened and closed manually by attaching a wrench to a head 15 extending from the top of the hydrant 10. When the valve 11 is opened, the hydrant 10 fills with water. Three externally threaded outlets 16a-c threaded into the vertical wall 17 of the hydrant 10 are capped with caps 19a-c (the cap 19a being removed and not shown). The caps 19a-c are individually manually removable, using a wrench. The outlets, illustratively and conventionally, include two 2.5″ NST male outlets 16a and 16c and one 4″ NST male outlet 16b. This construction is typical of a conventional fire hydrant, described for example in Ellis et al., U.S. Pat. Nos. 3,980,096 and 4,154,259.

The illustrative device 1 of the present invention is designed to be mounted to one of the 2.5″ NST outlets of the hydrant 10. It will be understood that a 4″ adapter may be provided to fit the standard 4″ outlet 16c of fire hydrant 10, if desired.

The adapter 3 illustratively includes a pipe length 31 having a 2-½″ NST female coupling 33 at one end and a 2-½″ NST male coupling 35 at its other end. The female coupling 33 of the adapter is rotatably connectable to the standard 2-½″ outlet 16a of the fire hydrant 10. The male end 35 of the adapter is attached to the proximal female connector 37 of a standard flushing hose 39.

Between the female end 33 and the male end 35 of the pipe length 31, a ⅜″ stub pipe 41 is threaded into the side of the wall of the pipe length 31. The free end of the stub pipe 41 is attached to a ball valve 43, which blocks flow when its handle 45 is turned to close the valve 43. It will be understood that the valve 43 may alternatively be formed to divert and expel water rather than block it.

A male quick-disconnect fitting 47 is threaded to the free end of the valve 43. The flexible sampling hose 7 is connected to the male fitting 47 by a female quick-disconnect fitting 49. The female fitting 49 is attached to the hose 7 with a standard hose barb.

The other end of flexible hose 7 includes a female quick-disconnect fitting 51, which may be identical to the fitting 49. The second female fitting 51 is chosen to engage a male quick-disconnect fitting 53, which may be identical to the fitting 47, on the box 5. The flushing hose 39 and sampling hose 7 are sized so that the sampling hose 7 carries a small fraction of the water carried by the flushing hose 39. The case 5 is conveniently carried from hydrant to hydrant in a vehicle such as a pickup truck, indicated at 55 in FIG. 1. At the site of the hydrant, the operator opens the tailgate 57 of the pickup truck, places the case 5 on the tailgate 57, optionally plugs the case 5 into a standard seven-way RV outlet 59 on a trailer hitch of the pickup truck, and snaps the sampling hose 7 to the fitting 53, all as described more fully hereinafter.

The box 5 contains a chlorine sensor 61, its associated flow cell 63, an analyzer 65, a programmable logic controller (PLC) 67, an alarm 69, a battery 71, and associated plumbing and electrical circuitry.

The box 5 is illustratively a hand-held portable carrying case having a molded plastic body 73, a lid 75 hinged to the body, and a carrying handle 77 attached to a front wall of the case 5. Also attached through the front wall are the fluid quick-disconnect 53, a hose fitting 79, and a small drain hose 80 attached to the hose fitting 79. Latches 81 hold the lid of the case shut when the device is not in use. The case 5 and its contents illustratively weigh less than thirty pounds; in this embodiment, they weigh about twenty-six pounds.

A plastic shelf 83 is hinged at 85 to the inside front of the case's body 73 and is positioned by a stop 87 at the inside back of the body 73. The analyzer 65, PLC 67, and alarm 69 are all mounted through the shelf 83, with their displays visible when the lid 75 of the case 5 is open, and with their electrical connections on the lower side of the shelf 83. A foam pad 89 in the lid 75 of the carrying case 5 holds the shelf 83 and its components in place and protects them against jostling when the case is being carried. The flow cell 63 and chlorine sensor 61 are mounted to the bottom of case 5 as described below and are visible through a cutout 91 in the shelf 83.

In this embodiment, the chlorine sensor 61, flow cell 63, and analyzer 65 are parts of an ATI Model Q45H-62 (Analytic Technology, Inc., Collegeville, Pa.) chlorine monitoring/recording system. Briefly, the sensor 61 is a membrane-covered polarographic sensor which measures chlorine directly. A pH input is obtained from a pH sensor 93 in a second flow cell 95, to give two-parameter monitoring and to adjust chlorine measurements for pH.

The PLC 67 in this embodiment is a Unitronics model Vision350 integrated Programmable Logic Controller and Human-Machine Interface (Unitronics Inc., Quincy, Mass.). The PLC 67 both monitors and records signals from the chlorine analyzer 65.

A DIN rail 101 mounted to the lower face of the shelf 83 carries terminal blocks 103 for connecting the electrical wiring of the components. Also mounted to the lower face of the shelf 83 is a DC/DC regulator 105 which maintains voltage levels within the specifications of the electronic components such as the analyzer 65 and PLC 67.

Mounted to the bottom wall of the case 5, below the shelf 83, and accessible when the shelf is lifted by hinging it forward, are the chlorine sensor 61, the pH sensor 93, a twenty-four volt lithium ion battery 71 having a built-in charge gauge, a charge controller 111 for controlling the charging of battery 71, a charging solenoid 113, and a fluid shutoff valve 115. The shutoff valve 115 includes an integral screen filter 117 to protect the sensors 61 and 93. A clamp 119 holds the shutoff valve 115 to the case body 73.

The fluid circuit components in the box 5 are shown, partially broken away, in FIG. 4. The quick-disconnect 53 is connected by a hose 131 to the shutoff valve 115 and filter 117, thence through a hose 133 to an inlet of the flow cell 95 of the pH sensor 93. An outlet of the pH sensor cell 95 is connected through a breather tee 136 by hose 135 to an inlet of the flow cell 63 of the chlorine sensor 61. The outlet of the flow cell 63 of chlorine sensor 61 is connected through a breather tee 137 to a hose 139, which is connected through the hose fitting 79 to external drain hose 80, thereby providing a drain for the flow cell 63.

The shutoff valve 115 permits servicing of the parts in the case body 73 without disconnecting the case from sampling hose 7. The bypass valve 43 in the sampling hose 7 removes major contaminants from the stream before sending water to the chlorine sensor 61 through the filter 117.

The power source for the monitor 1 may take any desired form. For example, the monitor may be powered entirely by an internal rechargeable battery which is taken out and recharged on a schedule. It is preferred, however, that power be taken from the pickup truck 55 through the standard trailer seven-way RV connector 59, either to power the monitor directly or to charge a battery in the monitor box 5. Seven-way connectors are well-know and are described, for example, in Orazem, U.S. Pat. No. 9,278,645. A cord grip or sealed electrical pass-through 161 is provided through a side wall of the case 5 for use with most of the alternative power sources. A cord 163 extending out of the cord grip 161 is provided with a connector 165 for attachment to circuitry plugged into a standard truck-mounted trailer seven-way RV connector 59. The connector 59 is electrically connected to the truck's battery, not shown.

A presently preferred power source is shown in FIG. 5. In this embodiment, power is supplied by the battery 71, and that battery is charged when the vehicle's engine is running; a separate charging circuit is also provided to enable charging from a 120-volt alternating current line source.

As shown in FIG. 5, the connector 165 connects the circuitry in the box 5 to a standard trailer seven-way RV connector 59. Power flows through the charging solenoid 113 which is activated when the vehicle engine is running, through a 12-volt DC to 24-volt DC converter 175, through the charge controller 111 and to lithium-ion battery 71. A second charging circuit is connected in parallel to the battery 71. This circuit may be plugged into a 120-volt outlet, through a 120 VAC to 24 VDC converter 177 which controls charging of the battery 71 when the second charging circuit is energized. The 120-volt charging circuit may be used periodically, such as every evening after a day's use when the operator does not have access to a vehicle with a seven-way RV adapter, or it may be used as a backup when the battery has discharged and needs a rapid charge. A manual switch 178 permits disconnecting the power supply from the circuitry in box 105 while the battery is removed or replaced.

In all the illustrative embodiments of the power source, power to the monitor circuitry may conveniently be provided through a plug 166 plugged into terminal block 103.

The electrical connections of the monitor circuitry are shown in FIG. 6.

Power is drawn through a plug 168 attached to the connector block 103. A cut-off switch 181 is provided in the power lines feeding the PLC, so that battery power may be conserved by powering down the PLC independent of the chlorine analyzer 65 and sensor 61, which require a longer warm-up period and are preferably left powered on during the day when the monitor 1 will be in use. If desired, the PLC, which has minimal power requirements, may also be continuously powered.

It will be understood that the PLC 67 may be programmed to send an alarm signal as soon as the pre-programmed chlorine level is reached, or it may be programmed to recheck the chlorine level at timed intervals to confirm that the level has been reached as discussed hereinafter. The PLC 67 may also be programmed to give a signal, either the same signal or a different signal, if flushing has proceeded beyond a predetermined time period.

In use, the operator approaches a hydrant 10 which is to be manually flushed, removes a protective cap 19a on the hydrant outlet, connects the adapter 3 and flushing hose 39 to the outlet, positions the box 5 as needed, keeping the box outlet hose 80 appropriately positioned, and connects the flexible sampling hose 7 to the box 5 with the quick-disconnect connector 51. Illustratively, the vehicle is a pickup truck 55, the case 5 is placed on the tailgate 57, and the electrical circuitry is connected to the vehicle battery by plugging the seven-way RV connector plug 165 into the jack 59 on the pickup truck.

The operator opens the lid 75 of the case 5, turns on the power-saving switch 181 if necessary, and follows the instructions that appear on the human-machine interface (HMI) of the PLC 67. The operator records in the PLC an identification of the hydrant 10, checks that the ball valve 43 is turned to block flow through the sampling hose 7, and manually opens the hydrant 10 using a wrench on the stem 15 of the main valve 11. After water has run through the flushing hose 39 sufficiently to clear any large debris or visible mud from the system, the diverter valve 43 is opened to allow water to flow through the sample hose 7. A small part of the water flowing through the adapter 3 is thus directed through the sample hose 7 through the filter 117 to the flow cell 63 of the chlorine sensor 61. The PLC 67 may log the start time it was turned on and/or the time it begins to receive signals from the analyzer 65.

The monitor 1 operates without further operator intervention. The analyzer 65 continuously sends a 4-20 mA signal to the PLC 67. The PLC 67 records the chlorine level represented by the signal, either continuously or periodically, against time until either a set chlorine level or a maximum time is reached. When the set chlorine level is reached, the PLC continues to monitor chlorine level for three seconds to ensure the level is maintained. If the level is maintained for three seconds, the alarm 69 is activated. If a maximum flush time is reached before the chlorine level reaches the set level, the alarm is also activated. When activated, the alarm 69 flashes and sounds to notify the operator that the hydrant should be closed.

When the alarm 69 is perceived, the operator presses an Acknowledge button on the HMI of the PLC 67, optionally takes a sample from the discharge hose 80, manually closes the main valve 11 of the hydrant 10, and presses a Stop button on the HMI of the PLC 67. The PLC 67 may log the time the alarm was activated, the time the Acknowledge button was pressed, and/or the time the Stop button was pressed. The operator then turns off the PLC 67 with the switch 181, unplugs the electrical connector 165, disconnects the sampling hose quick-disconnect 51 from the box quick-disconnect 53, disconnects the adapter 3 from the hydrant, rolls up the hoses 39 and 7, reattaches the cover of the hydrant outlet 16a, packs up the adapter 3 with its hoses 39 and 7, closes the box 5 and the tailgate 57, and proceeds to the next hydrant.

At the end of the day or week, the operator downloads data from the PLC onto a central computer and delivers the samples for lab testing. In embodiments, the data from the PLC is stored in a removable solid-state memory such as a micro SD card or a USB flash drive. In other embodiments, the PLC is SCADA enabled and transmits real-time data to a central computer, thereby eliminating the need to download the data manually. In a SCADA-enabled system, the central computer could monitor parameter levels and send an alarm signal when the monitored level(s) indicate the desired water quality. Such a system would permit, for example, several hydrants on a single line to be opened as part of a pipe cleaning operation, with the SCADA system monitoring multiple devices 1 and overseeing the operation.

It will be seen that the use of the manual flushing monitor device of the invention provides many of the benefits of automatic flushing devices and augments some of them, without the cost, complexity, and vulnerability to vandalism associated with automatic flushing devices. It also enhances and speeds up existing regular flushing programs and provides regular visual inspection of hydrants year-round. It further permits better understanding of the dynamics of the entire water system, by charting contamination and the extent of stagnant water, gauged by the absolute initial levels of water quality markers and the time to clearance, at locations throughout the system.

Numerous variations in the manual flushing monitor device of the present invention, within the scope of the appended claims, will immediately occur to those skilled in the art.

Merely by way of example, although the configuration shown in FIG. 1 is preferred because of its simplicity and ease of use, other configurations are possible. For example, the adapter 3 could be capped and attached to one outlet of the hydrant 10 while the flushing hose 39 is attached to a second outlet of the hydrant. The entire analyzer could be miniaturized and mounted directly to the hydrant 10 either on the same outlet as the hose 39 or on another outlet, although this approach is believed to be considerably more cumbersome for the operator.

An automatic time-delay valve may be substituted for the manual ball valve 43, or a large filter may be installed in the flushing hose 7.

The device may be divided in different ways; for example, the power supply could be in a different box.

The identity of each hydrant flushed may be automatically recorded by the use of a GPS sensor in the device. Telemetry capabilities, such as Bluetooth or other wireless communication with the operator's cellular telephone or other handheld or hands-free device, may be provided in the device for sending alarm signals (such as sounds or vibrations, or messages which generate notifications), for automatic uploading of data, or for SCADA control if desired. Samples may be automatically collected for more complete laboratory testing.

A simple data logger and appropriate circuitry with a limit setting may be used in place of the PLC, although the PLC gives far greater versatility.

The analyzer 65 may be eliminated if the sensor 61 provides a signal readable by the PLC 67. Newer sensors provide such signals. Eliminating the separate analyzer 65 allows the enclosure 5 to be smaller and lighter. The PLC 67 may display both battery charge and chlorine level, and may if so programmed, change its display to make those values more easily viewable at appropriate times during the flushing cycle.

Rather than a separate adapter attached between the hose and hydrant, a tap may be provided in the metal fitting of the hose for attachment of the flexible hose.

The alarm may take many alternative forms, including, for example, a wireless signal sent to a central monitoring station which in turn notifies the on-site operator, or a function built into the PLC or other logic circuit, such as changing the color of the PLC display, flashing the PLC display, or activating an internal PLC signal.

These variations are merely illustrative.

The disclosures of all patents, applications, and literature mentioned herein are hereby incorporated by reference.

1. A device for monitoring manual flushing of a hydrant, the device comprising an adapter constructed and proportioned to be attachable to an outlet of a water hydrant, an access port on the adapter, the access port being sized to take a sample of a stream of water flowing through the hydrant, a flexible hose attached to the access port, and an analyzer box containing a sensor adapted to sense a characteristic of water, the sensor being operatively connected to the flexible hose.

2. The device of claim 1 wherein the sensor is attached to an alarm, the alarm producing a signal when the characteristic reaches a predetermined level.

3. The device of claim 2 wherein the signal is audible.

4. The device of claim 2 wherein the signal is visible.

5. The device of claim 2 wherein the signal is conveyed through a portable electronic device.

6. The device of claim 1 further comprising a data logger attached to the sensor.

7. The device of claim 1 wherein the characteristic comprises a chlorine level in the water, the sensor comprising a chlorine sensor.

8. The device of claim 1 wherein the adapter is attached or attachable to a flushing hose.

9. The device of claim 1 wherein the device comprises a battery and a charging circuit for the battery.

10. The device of claim 9 wherein the sensor is a chlorine detector, the battery energizing the chlorine detector independent of energization of logic circuitry and logging circuitry.

11. A device for monitoring manual flushing of a hydrant, the device comprising an adapter constructed and proportioned to be attachable to an outlet of a water hydrant, an access port on the adapter, the access port being sized to take a sample of a stream of water flowing through the hydrant, a sensor attached to the access port, the sensor sensing a characteristic of water, a logic circuit attached to the sensor, the logic circuit producing a signal when the characteristic of the water reaches a predetermined level, and an alarm, the alarm being triggered in response to the signal to produce a humanly detectable alarm signal.

12. The device of claim 11 wherein the alarm signal is visible or audible or both.

13. The device of claim 11 wherein the sensor is a chlorine sensor.

14. The device of claim 11 further comprising a flushing hose for carrying most of the water from the hydrant.

15. A method of manually flushing a hydrant, the method comprising attaching an adapter to an outlet of the hydrant, the adapter being connected to an electronic water analyzer, opening a valve in the hydrant to expel water through the hydrant, capturing a portion of the water through the adapter and routing it to the analyzer, and manually closing the valve.

16. The method of claim 15 wherein the electronic water analyzer compares a water quality parameter with a preset value and generates a humanly detectable signal when the parameter reaches the preset value.

17. The method of claim 15 the electronic water analyzer electronically records a plurality of water quality parameter readings against time.
